(19) United States
(12) Patent Application Publication
(10) Pub. No.: US 2023/0321200 A1
(43) Pub. Date: Oct. 12, 2023

(54) SILK PARTICLES ENCAPSULATING OXYGEN CARRIERS AS ARTIFICIAL BLOOD SUBSTITUTE

(71) Applicant: University of Florida Research Foundation, Incorporated, Gainesville, FL (US)

(72) Inventors: Whitney L. STOPPEL, Gainesville, FL (US); Bruce Davis SPIESS, Gainesville, FL (US); Marisa PACHECO, Gainesville, FL (US)

(21) Appl. No.: 18/042,424

(22) PCT Filed: Aug. 20, 2021
(86) PCT No.: PCT/US2021/046888

§ 371 Date: Feb. 21, 2023

Related U.S. Application Data
(60) Provisional application No. 63/068,515, filed on Aug. 21, 2020.

(51) Int. Cl. A61K 38/42; A61K 9/16; A61K 31/02; A61K 41/00; A61K 9/107

(52) U.S. Cl. CPC A61K 38/42; A61K 9/1664; A61K 31/02; A61K 41/0028; A61K 9/1075; A61K 9/1641

(57) ABSTRACT

Described are silk fibroin nanoparticles encapsulating oxygen carriers and compositions containing silk fibroin nanoparticles encapsulating oxygen carriers. Also described are methods of using the silk fibroin nanoparticles encapsulating oxygen carriers as artificial blood substitutes.

### CROSS-REFERENCE TO RELATED APPLICATIONS

This application is a National Stage Application, filed under 35 U.S.C. § 371, of International Application No. PCT/US2021/046888, filed Aug. 20, 2021, which claims the benefit of U.S. Provisional Application No. 63/068,515, filed Aug. 21, 2020, each of which is incorporated herein by reference.

### STATEMENT REGARDING FEDERALLY SPONSORED RESEARCH OR DEVELOPMENT

This invention was made with government support under contract number W81XWH-21-1-0199, awarded by U.S. Army Medical Research Acquisition Activity. The government has certain rights in the invention.

### BACKGROUND

Donated blood is an important resource that is wasted for reasons that include deficiencies in its refrigeration, contamination with viruses from ongoing epidemics, and a short shelf-life of forty-two days. The cold-chain system and existing global health climate affect the viability of donated blood and other bioactive molecules.

The cold-chain system is used to safely transport and store vaccines, labile drugs, and donated blood [1, 2]. Keeping these compounds under tightly controlled temperatures is necessary but is not always accessible or affordable to people in developing or rural regions and to militants on the battlefield where hemorrhage is the main cause of preventable death [3,4]. According to the World Health Organization, the lack of proper resources and technology are major challenges that threaten donated blood safety [1].

The deficiencies in the cold-chain system account for viable drug losses adding to billions of dollars and for almost two percent of donated blood wastage [2]. Pandemics further threaten blood supply through infectious contamination in donated blood and limited access to healthy blood donors due to quarantine measures. Further, even with proper cold storage, there is a time-limit on donated blood, which can be stored only for up to forty-two days to preserve its oxygen-binding characteristics. The isolated red blood cell (RBC) component is adversely affected if stored longer than 14 to 21 days [5,6].

There is a need for a long-term and inexpensive method of transporting and storing oxygen carriers that mitigates the need for pathogen inactivation and the cold-chain system. The result would be useful for transfusion into patients with low red blood cell count in areas with limited resources and cold-chain technology.

### SUMMARY

Described are silk fibroin nanoparticles encapsulating oxygen carriers and compositions containing the silk fibroin nanoparticle particles encapsulating oxygen carriers. The oxygen carrier can be, but is not limited to, hemoglobin and perfluorocarbon. In some embodiments, the hemoglobin is a mammalian or a fish hemoglobin. In some embodiments, the hemoglobin is a Root effect hemoglobin. In some embodiments, the Root effect hemoglobin is a teleost hemoglobin. In some embodiments, the teleost hemoglobin is a salmon or trout hemoglobin. In some embodiments, the perfluorocarbon is a perfluorooctyl bromide. In some embodiments, the silk fibroin particles encapsulating the oxygen carrier are less than 1 μm in average or mean diameter. In some embodiments, the silk fibroin particles encapsulating the oxygen carrier are 200-500 nm in average or mean diameter.

Methods of making the silk fibroin nanoparticle particles encapsulating oxygen carriers are also described. The methods comprise degumming raw silk to form silk fibroin and encapsulating the oxygen carrier in silk fibroin nanoparticles formed by either lipid micelle encapsulation or phase separation. In some embodiments, lipid micelle encapsulation is performed using 1,2-dioleoyl-sn-glycero-3-phosphocholine or dipalmitoylphosphatidylcholine. In some embodiments, phase separation is performed using polyvinyl alcohol. In some embodiments, the silk fibroin particles formed by lipid micelle encapsulation or phase separation are sonicated. Sonication of the particles can be used to reduce or disrupt aggregation of particles and/or to generate smaller particles.

The silk fibroin particles encapsulating oxygen carriers or compositions containing the silk fibroin particles encapsulating oxygen carriers can be used as a blood substitute or as a component of a blood substitute. In some embodiments, the silk fibroin particles encapsulating oxygen carriers or compositions containing the silk fibroin particles encapsulating oxygen carriers are administering to the subject to increase oxygen supply to a tissue or organ in the subject. The silk fibroin particles encapsulating oxygen carriers or compositions containing the silk fibroin particles encapsulating oxygen carriers can also be used to perfuse an organ or tissue ex vivo.

Described are blood substitutes comprising an oxygen carrier encapsulated in silk fibroin particles. The silk fibroin particles can be less than 1000 nm in diameter, or about 200 to about 500 nm in diameter. The oxygen carrier can be hemoglobin, such as a Root effect hemoglobin, a teleost hemoglobin, a salmon hemoglobin, a trout hemoglobin, or a mammalian hemoglobin. The hemoglobin can also be a recombinant hemoglobin or a modified hemoglobin. The oxygen carrier can also be a perfluorocarbon, such as a perfluorooctyl bromide.

The described silk fibroin particles can be made from raw silk by degumming the raw silk. The size of the silk fibroin particles is affected to the degumming process. In some embodiments, the raw silk is degummed for 15-120 minutes or about 60 minutes.

The oxygen carrier can be encapsulated in the silk fibroin particles by phase separation or lipid micelle encapsulation methods. For phase separation, silk fibroin is added to the oxygen carrier in a polyvinyl alcohol (PVA) and water solution. The mixture is then sonicated and dried to form a film. The dried film is then dissolved in an aqueous solution to form silk fibroin particles encapsulating the oxygen carrier. In some embodiments, the oxygen carried is added to the silk particles after formation of the silk particles. In some embodiments, the resultant silk fibroin particles are further sonicated. The silk may be obtained from raw silk by degumming the raw silk for 15-120 minutes. The oxygen carrier can be hemoglobin such as a Root effect hemoglobin, a teleost hemoglobin, a salmon hemoglobin, a trout hemoglobin, or a mammalian hemoglobin. The hemoglobin can also be a recombinant hemoglobin or a modified hemoglobin. The oxygen carrier can also be a perfluorocarbon, such as a perfluorooctyl bromide. In some embodiments, the PVA and water solution contains 1-10% PVA, such as about 4-5% PVA. In some embodiments, the ratio of silk fibroin to the PVA and water solution is 0.1-10:100 weight to volume. In some embodiments the ratio of silk fibroin to PVA is 1:1-25 or about 1:4-5 weight to volume.

The size of the silk fibroin particles can be altered by adjusting the sonication amplitude (power) and time. In some embodiments, the silk fibroin solutions are sonicated for 5-60 seconds at 4-16 amplitude or at 8% to 50% power using a probe sonicator. In some embodiments, the silk fibroin solution is sonicated for 30 seconds at 12 amplitude or 25% power using a probe sonicator. In some embodiments, the silk particles are less than 1000 nm in diameter or about 200 to about 500 nm in diameter.

For lipid micelle encapsulation, a phospholipid film is formed on a surface. The phospholipid can be, but is not limited to, 1,2-dioleoyl-sn-glycero-3-phosphocholine or dipalmitoylphosphatidylcholine. Silk fibroin and the oxygen carrier in an aqueous solution are then added to the film to form lipid micelles. The lipid micelles are sonicated and subjected to at least one freeze-thaw cycle. The sonicated micelles are then lyophilized. After lyophilization the lipid micelles are washed with methanol to yield the silk fibroin particles encapsulating the oxygen carrier. The micelles may be further sonicated again prior to the at least one freeze-thaw cycle and/or prior to lyophilizing at step.

The size of the silk fibroin particles can be altered by adjusting the sonication amplitude (power) and time. In some embodiments, the silk fibroin solutions are sonicated for 5-60 seconds at 4-16 amplitude or at 8% to 50% power using a probe sonicator. In some embodiments, the silk fibroin solution is sonicated for 30 seconds at 12 amplitude or 25% power using a probe sonicator. In some embodiments, the silk particles are less than 1000 nm in diameter or about 200 to about 500 nm in diameter.

The silk fibroin particles encapsulating the oxygen carriers can be used in methods of treating a subject in need of increased oxygen supply to a tissue or organ. The silk fibroin particles encapsulating the oxygen carriers can be administered to a subject or used to perfuse a tissue or organ. In some embodiments, the subject is suffering from anemia, hemorrhage, acute loss of blood due to trauma or surgery, ischemia, hypoxia, or septic shock.

The described silk fibroin particles can be used to encapsulate other bioactive molecules. The described silk fibroin particles and methods of making and using the silk fibroin particles can be used for the encapsulation and controlled or sustained release of bioactive molecules.

### BRIEF DESCRIPTION OF THE DRAWINGS

FIG. 1. Diagram showing formulation of silk particles using phase separation between silk fibroin solution loaded with oxygen carriers and polyvinyl alcohol followed by sonication.

FIG. 2. Micrographs showing silk sponge formed using a lipid micelle method (left) and silk particles formed using a phase separation method and sonicated using a water bath sonicator (right).

FIG. 3. Micrographs showing particles resulting from the sonication of silk and PVA solution at 4 amplitude (upper left), 8 amplitude (upper right), 12 amplitude (lower left), and 16 amplitude (lower right).

FIG. 4. Scanning electron microscopy images of Control Sample (top left), Sample A (top right), Sample B (middle left), Sample C (middle right), Sample D (bottom left) and Sample E+F (bottom right).

FIG. 5A. Graph illustrating total frequency of particle diameters (μm) across all samples excluding Sample B. A cumulative percent line visually accounts for the percentage of particles graphed over time.

FIG. 5B. Graph illustrating frequency of particle diameter less than 1 μm across all samples excluding Sample B. A cumulative percent line visually accounts for the percentage of particles graphed over time.

FIG. 6. Micrographs showing bovine hemoglobin encapsulated in silk particles (top), silk particles auto fluoresce under blue light (bottom left), and bovine hemoglobin fluoresces in green due to secondary antibody (bottom right). Channels are split for easier observation of silk and hemoglobin individually.

FIG. 7. Light microscopy images of control sample (diluted) and Samples A-D taken at 60×. PFCs encapsulated in particles was observed as spheres that were opaquer than spheres in the diluted control.

FIG. 8A-H. Scanning electron microscopy images illustrating silk fibroin particle sizes generated by the phase separation method. (A-B) Particles formed using sonication at 8 amplitudes. (C-D) Particles formed using sonication at 12 amplitudes. (E-F) Particles formed using sonication at 14 amplitudes. (G-H) Particles formed using sonication at 16 amplitudes. (A, C, E, G) Particles after re-sonication. (B, D, F, H) Particles without re-sonication.

FIG. 9. Light microscopy images of silk fibroin particles analysis using ImageJ “Analyze Particles” tool.

FIG. 10. Scanning electron microscopy images illustrating silk fibroin particles.

FIG. 11. Light microscopy images of silk fibroin particles formed under different sonication amplitudes, numbers indicate % power using a probe sonicator.

FIG. 12A. Graph illustrating silk fibroin particle size as a function of sonication amplitude as determined by dynamic light scattering. Silk concentration was 5%. Silk to PVA ratio was 1:4. Silk degumming time was 60 minutes. Data are expressed as mean±1 standard deviation.

FIG. 12B. Graph illustrating silk fibroin particle size as a function of silk fibroin concentration as determined by dynamic light scattering. Silk to PVA ratio was 1:4. Silk degumming time was 60 minutes. Particles were sonicated at 25% power. Data are expressed as mean±1 standard deviation.

FIG. 12C. Graph illustrating silk fibroin particle size as a function of degumming time as determined by dynamic light scattering. Silk concentration was 5%. Silk to PVA ratio was 1:4. Particles were sonicated at 25% power. Data are expressed as mean±1 standard deviation.

FIG. 12D. Graph illustrating frequency of various particle sizes following sonication at 25% power.

FIG. 12E. Graph illustrating average particle size as a function of sonication amplitude.

FIG. 13. Graphs illustrating particle size distribution in samples sonicated at 8% power (top) and 40% power (bottom).

FIG. 14. Graph illustrating z-average particle size as a function of sonication amplitude.

FIG. 15. Scanning electron microscopy images illustrating silk fibroin particles formed at various sonication amplitudes.

FIG. 16. Graphs illustrating particle size distribution before (top) and after (bottom) filtration.

### DESCRIPTION

As used herein, including the appended claims, the singular forms of words such as “a,” “an,” and “the,” include their corresponding plural references unless the context clearly dictates otherwise. The conjunction “or” is to be interpreted in the inclusive sense, i.e., as equivalent to “and/or,” unless the inclusive sense would be unreasonable in the context. Use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

In general, the term “about” indicates insubstantial variation in a quantity of a component of a composition not having any significant effect on the activity or stability of the composition. The term “about” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed. All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.).

All references cited herein are incorporated by reference to the same extent as if each individual publication, patent application, or patent, was specifically and individually indicated to be incorporated by reference.

### I. Silk Fibroin

Silk fibroin is a thermodynamically stable, naturally derived protein fiber primarily derived from cocoon forming insects. Raw silk is composed of fibroin and sericin. Raw silk can be degummed to remove the sericin and yield silk fibroin. Silk fibroin is primarily hydrophobic with small hydrophilic pockets that form in between beta-sheets. Silk fibroin can be modified into several forms including, but not limited to, sponges, gels, and particles.

Silk fibroin is prepared from raw silk using methods known in the art. In some embodiments, silk fibroin is prepared from raw silk by degumming. Raw silk is degummed by incubating the raw silk in sodium carbonate in water. In some embodiments, the raw silk is incubated in 0.01-0.03 M sodium carbonate. In some embodiments, the raw silk is incubated in 0.02±0.01 M, 0.02±0.009 M, 0.02±0.008 M, 0.02±0.007 M, 0.02±0.005 M, 0.02±0.004 M, 0.02±0.003 M, 0.02±0.002 M, or 0.02±0.001 M sodium carbonate. In some embodiments, the raw silk is incubated in about 0.01 M, about 0.012 M, about 0.014 M, about 0.015 M, about 0.016 M, about 0.018 M, about 0.02 M, about 0.022 M, about 0.024 M, about 0.025 M, about 0.026 M, about 0.028 M, or about 0.03 M sodium carbonate. In some embodiments, the raw silk is incubated in about 0.02 M sodium carbonate. In some embodiments, the raw silk is degummed for 15 min to 2 hours. In some embodiments, the raw silk is degummed for 60±30 min, 60±25 min, 60±20 min, 60±15 min, 60±10 min, 60±5 min, or about 60 min. In some embodiments, the raw silk is degummed for 30±10 min, 30±5 min, 30±2 min, 30±1 min, or about 30 min. In some embodiments, the raw silk is degummed for 90±30 min, 90±25 min, 90±20 min, 90±15 min, 90±10 min, 90±5 min, or about 90 min. In some embodiments, the raw silk is degummed for about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, or about 90 minutes. Silk degumming time can affect the size of the silk fibroin. Longer degumming times lead to shorter fibroin proteins, while shorter degumming times yield longer fibroin proteins. In some embodiments, the raw silk is degummed in a sodium carbonate solution at about 90° C. to about 110° C. In some embodiments, the raw silk is degummed in a sodium carbonate solution at about 90° C., about 92° C., about 94° C., about 95° C., about 96° C., about 98° C., about 100° C., about 102° C., about 104° C., about 105° C., about 106° C., about 108° C., or about 110° C. The degummed silk is then washed to remove or substantial remove the sodium carbonate.

After degumming, the degummed silk is dried to yield silk mats. After drying, the silk mats are dissolved in a chaotropic agent solution, such as a lithium bromide solution. In some embodiments, the silk mats are dissolved in a solution that is about 8 M, about 8.1 M, about 8.2 M, about 8.3 M, about 8.4 M, about 8.5 M, about 8.6 M, about 8.7 M, about 8.8 M, about 8.9 M, about 9 M, about 9.1 M, about 9.2 M, about 9.3 M lithium bromide. In some embodiments, the silk mats are dissolved in 9.3 M lithium bromide. In some embodiments, the silk mats are dissolved in a lithium bromide solution at about 60° C. for about 4 hours. In some embodiments, the silk mats are dissolve in a lithium bromide solution at 40-80° C., 50-70° C., 60±10° C., 60±9° C., 60±8° C., 60±7° C., 60±6° C., 60±5° C., 60±4° C., 60±3° C., 60±2° C., 60±1° C., or 60° C. In some embodiments, the silk mats are dissolve in a lithium bromide solution at about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. In some embodiments, the silk mats are dissolved in a lithium bromide solution for 2-6 hours, 3-5 hours 4±2 hours, 4±1.75 hours, 4±1.5 hours, 4±1.25 hours, 4±1 hours, 4±0.75 hours, 4±0.5 hours, 4±0.25 hours, or about 4 hours. In some embodiments, the silk mats are dissolved in a lithium bromide solution for about 3 hours, about 3.25 hours, about 3.5 hours, about 3.75 hours, about 4 hours, about 4.25 hours, about 4.5 hours, about 4.75 hours, or about 5 hours. The lithium bromide is then removed or substantially removed. Removal of the lithium bromide can be by dialysis. Following degumming, the silk fibroin can be stored in aqueous solution.

In some embodiments, the silk fibroin is dissolved in aqueous solution. The silk fibroin can be dissolved in aqueous solution at 1-10% wt/volume. In some embodiments, the silk fibroin is dissolved in aqueous solution at about 1%, about 2%, about 3%, about 4%, about 5%, about 6%, about 7%, about 8%, about 9%, or about 10% wt/volume. In some embodiments, the silk fibroin is dissolved in aqueous solution at 4±3%, 4±2%, 4±1%, or about 4% wt/volume. In some embodiments, the silk fibroin is dissolved in aqueous solution at 5±3%, 5±2%, 5±1%, or about 5% wt/volume.

The silk fibroin can be, but is not limited to, Bombyx mori silk fibroin.

The silk fibroin can be prepared using good manufacturing practices. In some embodiments, the silk fibroin is prepared using current good manufacturing practices.

### II. Silk Fibroin Particles Encapsulating Oxygen Carriers

Silk fibroin is biocompatible and can be engineered as silk particles as a functional method to encapsulate and stabilize bioactive molecules. Silk fibroin particles encapsulating oxygen carriers can be formed by lipid micelle encapsulation or phase separation.

A. Lipid Micelle Encapsulation

In some embodiments, the silk fibroin particles are formed by lipid micelle encapsulation. For lipid micelle encapsulation, phospholipid is dissolved in an appropriate solvent and placed into a tube or other container. The solvent is then evaporated such that a lipid film is deposited on a surface of the container. Silk fibroin in aqueous solution and oxygen carrier is added to the film such that micelles spontaneously form. The silk fibroin can be obtained from raw silk as described above. In some embodiments, oxygen is dissolved in the perfluorocarbon prior to encapsulation. In some embodiments, hemoglobin is oxygenated prior to encapsulation. In some embodiments, the hemoglobin or perfluorocarbon is oxygenated after encapsulation. The spontaneously forming micelles encapsulate the silk fibroin. The micelles are then processed with 0-5 freeze-thaw cycles. In some embodiments, the micelles are processed with 1, 2, 3, 4, or 5 freeze-thaw cycles. In some embodiments, the micelles are processed with 3 freeze-thaw cycles. The micelle-containing solution is then diluted in water, frozen, and lyophilized. The lyophilized particles can be stored as a lyophilized product. After lyophilization, the lyophilized material is washed with methanol. In some embodiments, the particles are resolubilizing in methanol. Resolubilization in methanol can also be used to sterilize the particles.

The phospholipid can be, but is not limited to, 1,2-dioleoyl-sn-glycero-3-phosphocholine (DOPC) and dipalmitoylphosphatidylcholine (DPPC). In some embodiments, the phospholipid is DPOC. In some embodiments, the phospholipid is DPPC.

The solvent can be, but is not limited to chloroform. In some embodiments, the solvent is chloroform.

In some embodiments, the ratio of silk fibroin to aqueous solution is 1-10:100 wt/volume (i.e., 1-10%). In some embodiments, the silk fibroin is dissolved in aqueous solution at about 1%, about 2%, about 3%, about 4%, about 5%, about 6%, about 7%, about 8%, about 9%, or about 10% wt/volume. In some embodiments, the silk fibroin is dissolved in aqueous solution at 4±2%, 4±1%, or about 4% wt/volume. In some embodiments, the silk fibroin is dissolved in aqueous solution at about 4% wt/volume. In some embodiments, the silk fibroin is dissolved in aqueous solution at 5±2%, 5±1%, or about 5% wt/volume. In some embodiments, the silk fibroin is dissolved in aqueous solution at about 5% wt/volume

In some embodiments, the ratio of silk fibroin to oxygen carrier is 0.1-10:10 (wt/wt). In some embodiments, the ratio of silk fibroin to oxygen carrier is about 0.1:10, about 0.2:10, about 0.3:10, about 0.4:10, about 0.5:10, about 0.6:10, about 0.7:10, about 0.8:10, about 0.9:10, about 1:10, about 2:10, about 3:10, about 4:10, about 5:10, about 6:10, about 7:10, about 8:10, about 9:10, or about 10:10 (wt/wt). In some embodiments, the ratio of silk fibroin to oxygen carrier is about 1:10 (wt/wt).

The samples can be sonicated before the freeze-thaw cycles, after the freeze-thaw cycles but before lyophilization, after lyophilization, or a combination thereof. Sonication can be used to disrupt aggregation of particles and/or to generate smaller particles. In some embodiments, sonication power, amplitude, frequency, and/or duration are selected to produce particles that are less than about 1000 nm in average or mean diameter, less than about 900 nm in average or mean diameter, less than about 800 nm in average or mean diameter, less than about 700 nm in average or mean diameter, less than about 600 nm in average or mean diameter, or less than about 500 nm in average or mean diameter. Disruption of particle aggregation and measurement of particle size can be determined using methods known in the art, including, but not limited to, light microscopy, scanning electron microscopy, and light scattering analysis. Sonication can be performed using a probe sonicator. In some embodiments, sonication can be performed for 5-60 secs. In some embodiments, sonication is performed for about 5 sec, about 10 sec, about 15 sec, about 20 sec, about 25 sec, about 30 sec, about 35 sec, about 40 sec, about 45 sec, about 50 sec, about 55 sec, or about 60 sec. In some embodiments, sonication is performed for about 30±15 secs, about 30±10 secs, about 30±5 secs, or about 30 seconds. Sonication can be performed continuously over the indicated times and it can be performed in cycles of on and off. For example, sonication can be performed in cycles of 1 sec on and 1 sec off, 2 sec on and 2 sec off, 3 sec on and 3 sec off, 4 sec on and 4 sec off, 5 sec on and 5 sec off, 6 sec on and 6 sec off, 7 sec on and 7 sec off, 8 sec on and 8 sec off, 9 sec on and 9 sec off, 10 sec on and 10 sec off, or any combination of 1-10 sec on and 1-10 sec off. For sonication performed in cycles of on and off, the indicated sonication time can be the total on time or the total one and off time. Sonication can be performed at an amplitude setting of 4-16 using a QSonica700 sonicator with a ¼″ probe (i.e., 4-16 amplitude), or an equivalent setting using a different sonicator or probe. In some embodiments, sonication is performed at an amplitude setting of 4, 5, 6, 7, 8, 9, 10, 11, 12, 13, 14, 15, or 16 using a QSonica700 sonicator with a ¼″ probe. In some embodiments, sonication is performed at an amplitude setting of 8-16 using a QSonica700 sonicator with a ¼″ probe. In some embodiments, sonication is performed at an amplitude setting of 12-16 using a QSonica700 sonicator with a ¼″ probe. In some embodiments, sonication is performed at an amplitude setting of 8-12 using a QSonica700 sonicator with a ¼″ probe. In some embodiments, sonication is performed at an amplitude setting of 8 using a QSonica700 sonicator with a ¼″ probe. In some embodiments, sonication is performed at an amplitude setting of 12 (i.e., 12 amplitude) using a QSonica700 sonicator with a ¼″ probe. In some embodiments, sonication is performed at an amplitude setting of 16 using a QSonica700 sonicator with a ¼″ probe.

Alternatively, sonication can be performed at 5-50% power using a probe sonicator such as a QSonica700 sonicator with a ¼″ probe, or an equivalent setting using a different sonicator or probe. In some embodiments, sonication is performed at 5%, 6%, 7%, 8%, 9%, 10%, 11%, 12%, 13%, 14%, 15%, 16%, 17%, 18%, 19%, 20%, 25%, 30%, 35%, 40%, 45%, or 50% power using a QSonica700 sonicator with a ¼″ probe. In some embodiments, sonication is performed at about 8%, about 12%, about 15%, about 16%, about 25%, about 40%, or about 50% power using a QSonica700 sonicator with a ¼″ probe. In some embodiments, sonication is performed at about 8% power using a QSonica700 sonicator with a ¼″ probe. In some embodiments, sonication is performed at about 12% power using a QSonica700 sonicator with a ¼″ probe. In some embodiments, sonication is performed at about 16% power using a QSonica700 sonicator with a ¼″ probe. In some embodiments, sonication is performed at about 25% power using a QSonica700 sonicator with a ¼″ probe. In some embodiments, sonication is performed at about 40% using a QSonica700 sonicator with a ¼″ probe. In some embodiments, sonication is performed at about 50% using a QSonica700 sonicator with a ¼″ probe.

In some embodiments, the sonication is performed at 20±2 kHz, 20±1 kHz, or about 20 kHz. In some embodiments, the sample is maintained at about 0-5° C. during the sonication. In some embodiments, the sample in maintained on ice during the sonication.

“Sonication” is the act of applying sound energy to a sample to agitate the sample. An ultrasonic horn or probe is used to transmit ultrasonic vibrations to the sample being sonicated. The amplitude of the probe's vibrating surface is the distance between its position in the probe's fully extended and fully contracted states, and is measured in microns (μm). The amplitude can be adjusted from a generator's control panel. Different probes have different maximum amplitudes. Amplitude can be set to a certain level, which corresponds to a value in microns that can be identified using the horn's calibration certificate. The amplitude can be expressed in percent (%) or as an amplitude setting level. Once the amplitude is set, it will remain at the same level throughout the process, regardless of all other conditions. The probe vibrates at a frequency that can be measured in Hertz (Hz).

In some embodiments, the silk fibroin particles are 100-1000 nm particles. In some embodiments, the silk fibroin particles are about 1000 nm or less. In some embodiments, the silk fibroin particles are about 200 nm to about 900 nm, about 200 nm to about 800 nm, about 200 nm to about 700 nm, about 200 nm to about 600 nm, or 200 nm to about 500 nm. In some embodiments, the silk fibroin particles are smaller than mammalian red blood cells. In some embodiments, the silk fibroin particles are smaller than human red blood cells In some embodiments, the silk fibroin particles are smaller than the size that red blood cells are typically deformed into in vivo. In some embodiments, particle size can be altered by altering sonication amplitude, silk boiling (degumming) time, and/or ratio of silk fibroin to solution during particle formation.

In some embodiments, the silk fibroin particles are filtered to remove aggregates. In some embodiments, the silk fibroin particles are filtered to yield a more even particle size distribution (i.e., reduce particle size dispersity). In some embodiments, the silk fibroin particles are filtered to remove particles larger than about 1000 nm, larger than about 900 nm, larger than about 800 nm, larger than about 700 nm, larger than about 650 nm, larger than about 600 nm, or larger than about 500 nm. Filtration can be performed using methods known in the art. In some embodiments, filtration comprises centrifugal filtration. In some embodiments, filtration comprises centrifugal filtration using a 650 nm cutoff filter.

In some embodiments, the silk fibroin particles are prepared using good manufacturing practices. In some embodiments, the silk fibroin particles are prepared using current good manufacturing practices.

B. Phase Separation

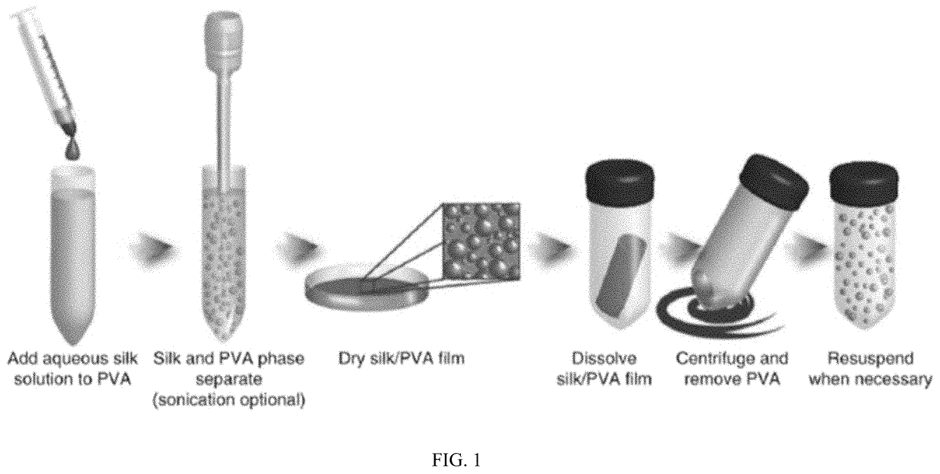

In some embodiments, the silk fibroin particles are formed by phase separation. Preparation of silk fibroin particles by phase separation is performed as illustrated in FIG. 1 and as described. Silk fibroin in aqueous solution and oxygen carrier are added to a solvent/water solution to form a fibroin/solvent/water solution. The silk fibroin can be obtained from raw silk as described above. In some embodiments, oxygen is dissolved in the perfluorocarbon prior to encapsulation. In some embodiments, hemoglobin is oxygenated prior to encapsulation. In some embodiments, the hemoglobin or perfluorocarbon are oxygenated after encapsulation. The fibroin/solvent/water solution is optionally sonicated. The fibroin/solvent/water solution is then placed into a container and dried to form a film. The film is then dissolved in aqueous solution, such as water. The resulting dissolved particles are then optionally sonicated before centrifugation to pellet the particles. After removal of the supernatant, the pelleted particles can be resuspended in an aqueous solution, such as water, and optionally sonicated, or stored until ready to use. To use, the pelleted particles are resuspended in aqueous solution, such as water, and optionally sonicated.

The solvent for use in formation of silk fibroin particle by phase separation comprises a liquid that is not readily soluble in water. In some embodiments, the solvent is an organic solvent. The organic solvent can be, but is not limited to, polyvinyl alcohol (PVA). In some embodiments, the solvent is polyvinyl alcohol (PVA).

The ratio (weight to volume) of solvent to water in the solvent/water solution can be, but is not limited to 1-10:100, 1:100, 1.5:100, 2:100, 2.5:100, 3:100, 3.5:100, 4:100, 4.5:100, 5:100, 5.5:100, 6:100, 6.5:100, 7:100, 7.5:100, 8:100, 8.5:100, 9:100, 9.5:100, or 10:100. In some embodiments, the solvent/water solution is a 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% solvent solution. In some embodiments, the solvent/water solution is a 1-10%, 2-10%, 3-10%, 4-10%, 5-10%, 1-9%, 2-9%, 3-9%, 4-9%, 5-9%, 1-8%, 2-8%, 3-8%, 4-8%, 5-8%, 1-7%, 2-7%, 3-7%, 4-7%, 5-7%, 1-6%, 2-6%, 3-6%, 4-6%, 5-6%, 1-5%, 2-5%, 3-5%, or 4-5% solvent solution. In some embodiments, the solvent/aqueous solution is about 5% solvent. In some embodiments, the solvent/aqueous solution is about 4% solvent. In some embodiments, the phase separation solution is 1-10%, 2-10%, 3-10%, 4-10%, 5-10%, 1-9%, 2-9%, 3-9%, 4-9%, 5-9%, 1-8%, 2-8%, 3-8%, 4-8%, 5-8%, 1-7%, 2-7%, 3-7%, 4-7%, 5-7%, 1-6%, 2-6%, 3-6%, 4-6%, 5-6%, 1-5%, 2-5%, 3-5%, or 4-5% solvent. In some embodiments, the phase separation solution is about 5% solvent. In some embodiments, the phase separation solution is about 4% solvent.

The ratio (weight to volume) of PVA to aqueous solution in the PVA/aqueous solution can be, but is not limited to, 1-10:100, 1:100, 1.5:100, 2:100, 2.5:100, 3:100, 3.5:100, 4:100, 4.5:100, 5:100, 5.5:100, 6:100, 6.5:100, 7:100, 7.5:100, 8:100, 8.5:100, 9:100, 9.5:100, or 10:100. In some embodiments, the PVA/aqueous solution is a 1%, 1.5%, 2%, 2.5%, 3%, 3.5%, 4%, 4.5%5, 5% 5.5%, 6%, 6.5%, 7%, 7.5%, 8%, 8.5%, 9%, 9.5%, or 10% PVA solution. In some embodiments, the PVA/aqueous solution is a 1-10%, 2-10%, 3-10%, 4-10%, 5-10%, 1-9%, 2-9%, 3-9%, 4-9%, 5-9%, 1-8%, 2-8%, 3-8%, 4-8%, 5-8%, 1-7%, 2-7%, 3-7%, 4-7%, 5-7%, 1-6%, 2-6%, 3-6%, 4-6%, 5-6%, 1-5%, 2-5%, 3-5%, or 4-5% PVA solution. In some embodiments, the PVA/aqueous solution is about 5% PVA. In some embodiments, the PVA/aqueous solution is about 4% PVA. In some embodiments, the phase separation solution is 1-10%, 2-10%, 3-10%, 4-10%, 5-10%, 1-9%, 2-9%, 3-9%, 4-9%, 5-9%, 1-8%, 2-8%, 3-8%, 4-8%, 5-8%, 1-7%, 2-7%, 3-7%, 4-7%, 5-7%, 1-6%, 2-6%, 3-6%, 4-6%, 5-6%, 1-5%, 2-5%, 3-5%, or 4-5% PVA. In some embodiments, the phase separation solution is about 5% PVA. In some embodiments, the phase separation solution is about 4% PVA.

The ratio of silk fibroin to solvent/water solution can be, but is not limited to, 0.1-10:100 weight to volume. In some embodiments, the ratio of silk fibroin to solvent/water solution is about 0.1:100, about 0.2:100, about 0.3:100, about 0.3:100, about 0.4:100, about 0.5:100, about 0.6:100, about 0.7:100, about 0.8:100, about 0.9:100, about 1:100, about 1.1:100, about 1.2:100, about 1:3:100, about 1.4:100, about 1.5:100, about 2:100, about 2.5:100, about 3:100, about 3.5:100, about 4:100, about 4.5:100, about 5:100, about 5.5:100, about 6:100, about 6.5:100, about 7:100, about 7.5:100, about 8:100, about 8.5:100, about 9:100, about 9.5:100, or about 10:100 weight to volume.

The ratio of silk fibroin to solvent can be 1:1-25 weight to volume. In some embodiments, the ratio of silk fibroin to solvent is about 1:1, about 1:2, about 1:3, about 1:4, about 1:5, about 1:6, about 1:7, about 1:8, about 1:9, about 1:10, about 1:12, about 1:15, about 1:20, or about 1:25 weight to volume.

In some embodiments, the ratio of silk fibroin to oxygen carrier is 0.1-10:10 (wt/wt). In some embodiments, the ratio of silk fibroin to oxygen carrier is about 0.1:10, about 0.2:10, about 0.3:10, about 0.4:10, about 0.5:10, about 0.6:10, about 0.7:10, about 0.8:10, about 0.9:10, about 1:10, about 2:10, about 3:10, about 4:10, about 5:10, about 6:10, about 7:10, about 8:10, about 9:10, or about 10:10 (wt/wt). In some embodiments, the ratio of silk fibroin to oxygen carrier is about 1:10 (wt/wt).

In some embodiments, the phase separation is carried out using a silk fibroin concentration of about 20 mg/mL to about 70 mg/mL. The concentration of silk fibroin can be about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, or about 70 mg/mL.

Sonication can be used to disrupt particle aggregation and/or generate smaller particles. In some embodiments, sonication power, amplitude, frequency, and/or duration are selected to disrupt particle aggregation and/or produce particles that are less than about 1000 nm in average or mean diameter, less than about 900 nm in average or mean diameter, less than about 800 nm in average or mean diameter, less than about 700 nm in average or mean diameter, less than about 600 nm in average or mean diameter, or less than about 500 nm in average or mean diameter. Disruption of particle aggregation and measurement of particle size can be determined using methods known in the art, including, but not limited to, light microscopy, scanning electron microscopy, and light scattering analysis. Sonication can be performed using a probe sonicator. In some embodiments, sonication can be performed for 5-60 secs. In some embodiments, sonication is performed for about 5 sec, about 10 sec, about 15 sec, about 20 sec, about 25 sec, about 30 sec, about 35 sec, about 40 sec, about 45 sec, about 50 sec, about 55 sec, or about 60 sec. In some embodiments, sonication is performed for about 30±15 secs, about 30±10 secs, about 30±5 secs, or about 30 seconds. Sonication can be performed continuously over the indicated times and it can be performed in cycles of on and off. For example, sonication can be performed in cycles of 1 sec on and 1 sec off, 2 sec on and 2 sec off, 3 sec on and 3 sec off, 4 sec on and 4 sec off, 5 sec on and 5 sec off, 6 sec on and 6 sec off, 7 sec on and 7 sec off, 8 sec on and 8 sec off, 9 sec on and 9 sec off, 10 sec on and 10 sec off, or any combination of 1-10 sec on and 1-10 sec off. For sonication performed in cycles of on and off, the indicated sonication time can be the total on time or the total one and off time. Sonication can be performed at an amplitude setting of 4-16 using a QSonica700 sonicator with a ¼″ probe (i.e., 4-16 amplitude), or an equivalent setting using a different sonicator or probe. In some embodiments, sonication is performed at an amplitude setting of 4, 5, 6, 7, 8, 9, 10, 11, 12, 13, 14, 15, or 16 using a QSonica700 sonicator with a ¼″ probe. In some embodiments, sonication is performed at an amplitude setting of 8-16 using a QSonica700 sonicator with a ¼″ probe. In some embodiments, sonication is performed at an amplitude setting of 12-16 using a QSonica700 sonicator with a ¼″ probe. In some embodiments, sonication is performed at an amplitude setting of 8-12 using a QSonica700 sonicator with a ¼″ probe. In some embodiments, sonication is performed at an amplitude setting of 8 using a QSonica700 sonicator with a ¼″ probe. In some embodiments, sonication is performed at an amplitude setting of 12 (i.e., 12 amplitude) using a QSonica700 sonicator with a ¼″ probe. In some embodiments, sonication is performed at an amplitude setting of 16 using a QSonica700 sonicator with a ¼″ probe.

Sonication can be performed at 5-50% power using a probe sonicator, such a QSonica700 sonicator with a ¼″ probe, or an equivalent setting using a different sonicator or probe. In some embodiments, sonication is performed at 5%, 6%, 7%, 8%, 9%, 10%, 11%, 12%, 13%, 14%, 15, 16%, 17%, 18%, 19%, 20%, 25%, 30%, 35%, 40%, 45%, or 50% power using a QSonica700 sonicator with a ¼″ probe. In some embodiments, sonication is performed at about 8%, about 12%, about 15%, about 16%, about 25%, about 40%, or about 50% power using a QSonica700 sonicator with a ¼″ probe. In some embodiments, sonication is performed at about 8% power using a QSonica700 sonicator with a ¼″ probe. In some embodiments, sonication is performed at about 12% power using a QSonica700 sonicator with a ¼″ probe. In some embodiments, sonication is performed at about 16% power using a QSonica700 sonicator with a ¼″ probe. In some embodiments, sonication is performed at about 25% power using a QSonica700 sonicator with a ¼″ probe. In some embodiments, sonication is performed at about 40% using a QSonica700 sonicator with a ¼″ probe. In some embodiments, sonication is performed at about 50% using a QSonica700 sonicator with a ¼″ probe.

In some embodiments, the sonication is performed at 20±2 kHz, 20±2 kHz, or about 20 kHz. In some embodiments, the sample in maintained at about 0-5° C. during the sonication. In some embodiments, the sample in maintained on ice during the sonication.

In some embodiments, the silk fibroin particles are 100-1000 nm particles. In some embodiments, the silk fibroin particles are about 1000 nm or less. In some embodiments, the silk fibroin particles are about 200 nm to about 900 nm, about 200 nm to about 800 nm, about 200 nm to about 700 nm, about 200 nm to about 600 nm, or 200 nm to about 500 nm. In some embodiments, the silk fibroin particles are smaller than mammalian red blood cells. In some embodiments, the silk fibroin particles are smaller than human red blood cells In some embodiments, the silk fibroin particles are smaller than the size that red blood cells are typically deformed into in vivo. In some embodiments, particle size can be altered by altering sonication amplitude, silk boiling (degumming) time, and/or ratio of silk fibroin to solution during particle formation.

In some embodiments, the silk fibroin particles are filtered to remove aggregates. In some embodiments, the silk fibroin particles are filtered to yield a more even particle size distribution (i.e., reduce particle size dispersity). In some embodiments, the silk fibroin particles are filtered to remove particles larger than about 1000 nm, larger than about 900 nm, larger than about 800 nm, larger than about 700 nm, larger than about 650 nm, larger than about 600 nm, or larger than about 500 nm. Filtration can be performed using methods known in the art. In some embodiments, filtration comprises centrifugal filtration. In some embodiments, filtration comprises centrifugal filtration using a 650 nm cutoff filter.

In some embodiments, the silk fibroin particles are prepared using good manufacturing practices. In some embodiments, the silk fibroin particles are prepared using current good manufacturing practices.

C. Phase Separation, Ouzo Method (Sample B)

Perfluorocarbons (PFCs) have a very low solubility in water and can be thermodynamically forced out of a hydrophilic solution to form nanodroplets [16]. This property can be enhanced using the Ouzo method in which ethanol mixed with water is used to decrease the solubility of PFCs [18].

### III. Oxygen Carrier

Oxygen carriers include perfluorocarbons (PFCs) and naturally occurring oxygen carriers. A naturally occurring oxygen carrier can be, but is not limited to, a hemoglobin. Oxygen can be dissolved in perfluorocarbons or bound to hemoglobin proteins. Both PFCs and hemoglobin protein can dissolve oxygen in high quantities.

“Hemoglobin” is a protein that normally contained within a red blood cell that transports oxygen. Each molecule of hemoglobin has 4 subunits, 2 α chains and 2 β chains, arranged in a tetrameric structure. A hemoglobin may be a naturally occurring hemoglobin or a synthetic hemoglobin. Hemoglobin may be isolated and purified from an animal or cell, or may be produced by chemical synthesis.

“Modified hemoglobin” includes, but is not limited to, hemoglobin altered by a chemical reaction such as intra- and inter-molecular cross-linking, genetic manipulation, polymerization, and/or conjugation to other chemical groups. A hemoglobin is “modified” if any of its structural or functional properties have been altered from its native state.

“Perfluorocarbons” (PFCs) are organofluorine compounds with the formula CₓF_{y}, but may have one or more heteroatoms, such as, but not limited to bromine.

In some embodiments, the oxygen carrier is a perfluorocarbon. For oxygen dissolved in PFCs, the relationship between dissolved oxygen and P_{O2} is linear. The perfluorocarbon can be, but is not limited to, perfluorooctyl bromide.

In some embodiments, the oxygen carrier is a hemoglobin. In some embodiments, the hemoglobin is a pH sensitive hemoglobin. In some embodiments, pH sensitive hemoglobin is a Root effect hemoglobin, i.e., the hemoglobin binds and releases oxygen following the Root effect. For Root effect hemoglobins, an increase in proton or carbon dioxide (lower pH) lowers the hemoglobin's affinity and carrying capacity for oxygen. Hemoglobins showing a Root effect exhibit a loss of cooperativity at low pH. Hemoglobin that binds to oxygen following the Root effect has an enhanced ability to release oxygen to tissues (Rummer et al. 2015). In some embodiments, the hemoglobin releases oxygen as a function of partial pressure, pH, and/or iron coordination. In some embodiments, the hemoglobin comprises a piscine (fish) hemoglobin. In some embodiments, the hemoglobin comprises a teleost hemoglobin. A teleost hemoglobin can be, but is not limited to, salmon hemoglobin and trout hemoglobin. Salmon hemoglobin and trout hemoglobin bind oxygen following the Root effect.

In some embodiments, the hemoglobin is mammalian hemoglobin. Mammalian hemoglobin can be, but is not limited to, human hemoglobin, bovine hemoglobin, horse hemoglobin, pig hemoglobin, sheep hemoglobin, and goat hemoglobin.

In some embodiments, the hemoglobin is recombinant, synthetic, or modified hemoglobin. In some embodiments, the hemoglobin contains one or more amino acid substitutions, deletions, or insertions that alter affinity, carrying capacity, or oxygen carrying pH sensitivity of the hemoglobin.

### IV. Methods of Use

The described silk fibroin particles encapsulating oxygen carriers (particles) can be used as artificial blood substitutes. In some embodiments, the described particles can be used as a substitutes for red blood cells. In some embodiments, the described particles are used to form compositions that can be used as a blood substitute or as a component of a blood substitute.

In some embodiments, a compositions containing any of the described silk fibroin particles encapsulating oxygen carriers may be used to supplement the oxygen-carrying capacity of a subject's blood.

In some embodiments, a composition containing any of the described silk fibroin particles encapsulating oxygen carriers are administered to a subject in need of blood. In some embodiments, compositions containing the described silk fibroin particles encapsulating oxygen carriers are administered to a subject to supplement the oxygen-carrying capacity of a subject's blood. In some embodiments, compositions containing the described silk fibroin particles encapsulating oxygen carriers are used to administer oxygen carrying nanoparticles into a subject.

In some embodiments, a composition containing any of the described silk fibroin particles encapsulating oxygen carriers may be administered to a subject to deliver oxygen to an organ or tissue.

In some embodiments, a composition containing any of the described silk fibroin particles encapsulating oxygen carriers are administered to a subject suffering from anemia, hemorrhage, acute loss of blood due to trauma or surgery, ischemia, hypoxia, or septic shock.

In some embodiments, methods of supplementing the oxygen-carrying capacity of a subject's blood are described comprising administering to the subject a composition containing any of the described silk fibroin particles encapsulating oxygen carriers. In some embodiments, the subject is in need of restoration of oxygen levels or increased oxygen levels.

In some embodiments, methods of delivering oxygen to a tissue or organ in a subject are described comprising administering to the subject a composition containing any of the described silk fibroin particles encapsulating oxygen carriers.

In some embodiments, compositions containing the described silk fibroin particles encapsulating oxygen carriers can be used to conduct blood transfusion in a patient, perfuse an organ or tissue in situ, perfuse an organ or tissue ex vivo, or to supply oxygen to cells either in situ or ex vivo.

The subject can be, but is not limited to, a mammal, an amphibian, a reptile, a bird, and a fish. Mammals include, but are not limited to, humans, horses, cats and dogs.

### REFERENCES

1. Mvere D et al. The blood cold chain: guide to the selection and procurement of equipment and accessories. (Department of Blood Safety and Clinical Technology, World Health Organization, 2002).
2. Zhang, J. et al. Stabilization of vaccines and antibiotics in silk and eliminating the cold chain. Proceedings of the National Academy of Sciences 109, 11981, doi:10.1073/pnas.1206210109 (2012).
3. Ashok A et al. Improving cold chain systems: Challenges and solutions. Vaccine 35, 2217-2223, doi:10.1016/j.vaccine.2016.08.045 (2017).
4. Gurney J M et al. Blood transfusion management in the severely bleeding military patient. Curr Opin Anaesthesiol 31, 207-214, doi: 10.1097/aco.0000000000000574 (2018).
5. Zallen G. et al. Age of transfused blood is an independent risk factor for postinjury multiple organ failure. The American Journal of Surgery 178, 570-572, doi:https://doi.org/10.1016/S0002-9610(99)00239-1 (1999).
6. Sparrow R L. Red Blood Cell Storage Duration and Trauma. Transfusion Medicine Reviews 29, 120-126, doi:https://doi.org/10.1016/j.tmrv.2014.09.007 (2015).
7. Rockwood D N et al. Materials fabrication from Bombyx mori silk fibroin. Nature protocols 6, 1612-1631, doi:10.1038/nprot.2011.379 (2011).
8. Kundu B et al. Silk proteins for biomedical applications: Bioengineering perspectives. Progress in Polymer Science 39, 251-267, doi:https://doi.org/10.1016/j.progpolymsci.2013.09.002 (2014).
9. Lucke M et al. Engineered hybrid spider silk particles as delivery system for peptide vaccines. Biomaterials 172, 105-115, doi:https://doi.org/10.1016/j.biomaterials.2018.04.008 (2018).
10. Zhang J et al. Silk particles, microfibres and nanofibres: A comparative study of their functions in 3D printing hydrogel scaffolds. Materials Science and Engineering: C 103, 109784, doi:https://doi.org/10.1016/j.msec.2019.109784 (2019).
11. Wang X et al. Silk nanospheres and microspheres from silk/pva blend films for drug delivery. Biomaterials 31, 1025-1035, doi:10.1016/j.biomaterials.2009.11.002 (2010).
12. Zhao Z et al. Silk fibroin-based nanoparticles for drug delivery. International journal of molecular sciences 16, 4880-4903, doi:10.3390/ijms16034880 (2015).
13. Carissimi G et al. Revealing the Influence of the Degumming Process in the Properties of Silk Fibroin Nanoparticles. Polymers (Basel) 11, doi:10.3390/polym11122045 (2019).
14. Spiess B D Perfluorocarbon emulsions as a promising technology: a review of tissue and vascular gas dynamics. Journal of Applied Physiology 106, 1444-1452, doi:10.1152/japplphysiol.90995.2008 (2009).
15. Roos R W et al. 1D Measurement of Sodium Ion Flow in Hydrogel After a Bath Concentration Jump. Annals of Biomedical Engineering 43, 1706-1711, doi:http://dx.doi.org/10.1007/s10439-015-1293-8 (2015).
16. Spiess B D Perfluorocarbon Gas Transport: an Overview of Medical History With Yet Unrealized Potentials. Shock 52, 7-12, doi:10.1097/shk.0000000000001150 (2019).
17. Rummer J L et al. Root Effect Haemoglobins in Fish May Greatly Enhance General Oxygen Delivery Relative to Other Vertebrates. PloS one 10, e0139477-e0139477, doi:10.1371/joumal.pone.0139477 (2015).
18. Li D S et al. Polypyrrole-Coated Perfluorocarbon Nanoemulsions as a Sono-Photoacoustic Contrast Agent. Nano letters 17, 6184-6194, doi:10.1021/acs.nanolett.7b02845 (2017).
19. Lu M et al. Preparation, characterization and in vivo investigation of blood-compatible hemoglobin-loaded nanoparticles as oxygen carriers. Colloids Surf B Biointerfaces 139, 171-179, doi:10.1016/j.colsurfb.2015.12.012 (2016).
20. McCall R L et al. “PLGA nanoparticles formed by single- or double-emulsion with vitamin E-TPGS.” Journal of visualized experiments: JoVE, 51015-51015, doi:10.3791/51015 (2013).

### EXAMPLES

### Example 1. Isolation of Silk Fibroin (Silk Degumming)

Silk cocoons were cut into dime-sized pieces. 5 grams of cut cocoons were degummed in 0.02 M of sodium carbonate in dH₂O (Sigma Aldrich, product no. 451614) for 60 minutes. The resultant silk mats was rinsed 3× to remove traces of sodium carbonate and dried under the fume hood for 48 hours. After drying completely, silk mats were completely dissolved in 9.3 M lithium bromide (Sigma Aldrich, product no. 213225) solution at 60° C. for four hours. Lithium bromide was removed by loading dissolved silk solution into dialysis membrane tubing (Thermo Scientific, product no. 88244) and dialyzing against several changes of deionized water, with stirring, over the next 72 h (e.g., changed at one, three, and five-hour intervals within the first 24 hours of dialysis and in the morning, afternoon, and night for the next 48 hours). The silk fibroin was stored at 4° C. for up to one month.

The concentration of the final aqueous silk fibroin solution was averaged using three samples and determined using the following formula:

$$\% \frac{wt}{vol} = \frac{Mass of 0.5 mL dried silk}{0.5}$$

### Example 2. Silk Fibroin Particle Formation

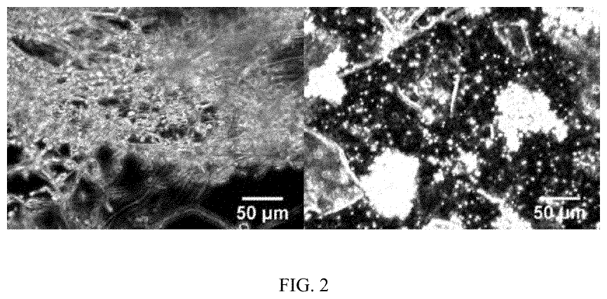

Preliminary experiments were performed to demonstrated silk fibroin particle formation using a lipid micelle encapsulation method or a phase separation method. Silk fibroin particles made using phase separation method were sonicated using a water bath sonicator. As shown in FIG. 2 (left image), in the absence of sonication, the lipid micelle encapsulation method generated a sponge. Also as shown in FIG. 2 (right image), the phase separation method results in silk fibroin particles that were aggregated. The lipid micelle encapsulation method resulted in formation of a silk fibroin sponge after lyophilization rather than particles. This result suggest the silk fibroin solution may not have been adequately mixed with the lipid to form micelles.

### Example 3. Silk Particles Formed by Lipid Encapsulation

Glass test tubes were coated in phospholipid by dissolving dipalmitoylphosphatidyl-choline (Fisher Scientific, product no. NC7467752) in 1 mL of chloroform under a fume hood. The chloroform was dried using direct nitrogen gas flow through a 0.5 cm diameter tube. The tubes were rolled and as the chloroform evaporated, lipid residue was left on the sides of the test tubes. Aqueous silk solution with or without oxygen carrier was diluted to 4% wt/vol with deionized water and slowly added to the tubes, allowing the lipid to mix and ensuring all or substantially all of the lipid is removed from the sides of the tube. Lipid micelles encapsulating the aqueous solution are spontaneously formed during the mixing. Deionized water was added to the solution for complete dissolution before transferring the mixtures to a 15 mL plastic conical tubes. Three freeze-thaw cycles were completed to obtain a more homogenous distribution of particles by submerging the conical tubes into liquid nitrogen for fifteen minutes followed by another 15 minutes of submersion in a 37° C. water bath. After the last thawing, silk/lipid solution was slowly pipetted into 100 mL of deionized water fast-stirring on a stir-plate. The solution was transferred to 50 mL conical tubes and frozen at −80° C. overnight. Frozen solution was lyophilized for three days. After lyophilization, 30 mg of lyophilized product was transferred into a large 2-ml microcentrifuge tube and 2 mL of pure methanol was added and incubated at room temperature for 30 minutes. The soluble portion of the solution was decanted and centrifuged at 10,058 rpm for five minutes at 4° C.

Subsequently, the methanol is discarded and the pellet is dried in a fume hood overnight. The pellet is then washed with methanol and the particles resuspended in deionized water.

### Example 4. Forming Silk Particles by Phase Separation

A 5% wt/vol polyvinyl alcohol (PVA) (Sigma Aldrich, product no. P8136) was made by dissolving PVA in milli-Q water at 60° C. overnight. Solution was sterilized using a 0.33 μm or 0.45 μm syringe filter. For standard samples, 4 mL of sterilized 5% PVA solution was added to 1 mL of 4% wt/vol silk solution with or without oxygen carrier. Solutions were sonicated at various amplitude (QSonica700) using a ¼″ microtip at different amplitude settings for 30 seconds. Sonication at an amplitude of 12 amp for 30 sec was used in the following experiments. Samples were cast in petri dishes and left to dry completely, partially uncovered and under a Kimwipe inside the fume hood. Once dried, films were peeled off the petri dish and dissolved in 20 mL milli-Q water. Dissolved solution was transferred to 2 mL microcentrifuge tubes and centrifuged at 3000-10770 rpm for 20 minutes at 4° C. Supernatant was discarded and the pellets were resuspended in 5 mL milli-Q water. Particle aggregates were broken apart using a second sonication of at varying amplitudes (8, 12, 14, 14 or 16 amplitudes) for 15 seconds (FIG. 1).

In some embodiments, the particles are re-sonicated. In some embodiments, re-sonication is performed for 15±10 seconds, 15±8 seconds, 15±6 seconds, 15±5 seconds, 15±4 seconds, 15±3 seconds, 15±2 seconds, 15±1 seconds, or 15 seconds. In some embodiments, re-sonication is performed at an amplitude of 4, 5, 6, 7, 8, 9, 10, 11, 12, 13, 14, 15, or 16 using a QSonica700 sonicator with a ¼″ probe. In some embodiments, re-sonication is performed at 15 amplitudes for 15 seconds.

In some embodiments, the particles are filtered to further remove aggregates and yield a more even particle size distribution.

### Example 5. Effect of Sonication Amplitude on Particle Size

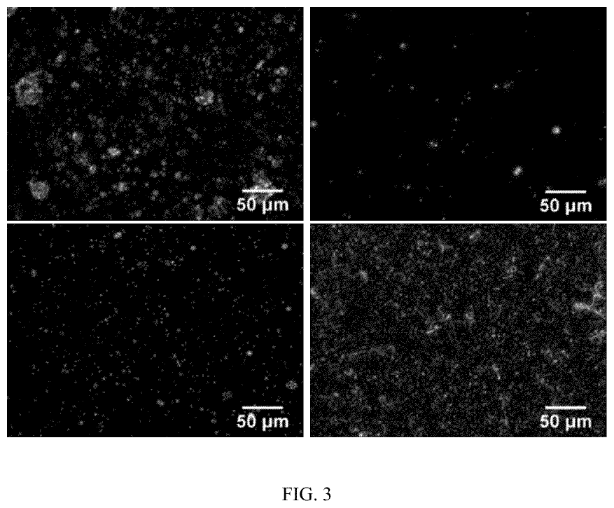

Silk fibroin particles formed by phase separation were sonicated at varying amplitude settings to disrupt aggregation of particles and to generate smaller particles. The silk fibroin particles were sonicated using a QSonica700 sonicator with a ¼″ microtip. FIG. 3 shows particles resulting from the sonication of PVA/silk solution at 4 amplitude (upper left), 8 amplitude (upper right), 12 amplitude (lower left), and 16 amplitude (lower right). Sonication disrupted aggregation or particles and resulted in particles having smaller size. Samples sonicated at 4 amplitude yielded particles with some aggregation even after a second sonication step. Samples sonicated at 8 amplitude yielded small particles without significant aggregation. Samples sonicated at 12 amplitude yielded more small particles than the samples sonicated at 8 amplitude. Samples subjected to sonication at 16 amplitude had fewer visible particles.

Images were uploaded to ImageJ (NIH). The threshold was adjusted to contrast lighter colored particles against a dark background. Using “Analyze Particles,” images were sorted by particle diameters of 0.001 nm-1000 nm. Results were exported to Microsoft Excel where the data was binned into frequency of particles within specific ranges of diameters and graphically represented using histograms. ImageJ analysis of samples sonicated at 8 amplitude and 12 amplitude showed similar polydispersity as shown in Table 1. The smallest measurement for these particles was one micron since the images were taken under light microscopy.

Samples sonicated at 12 amplitude resulted in the smallest particles on average and had the most particles present that were not aggregated.

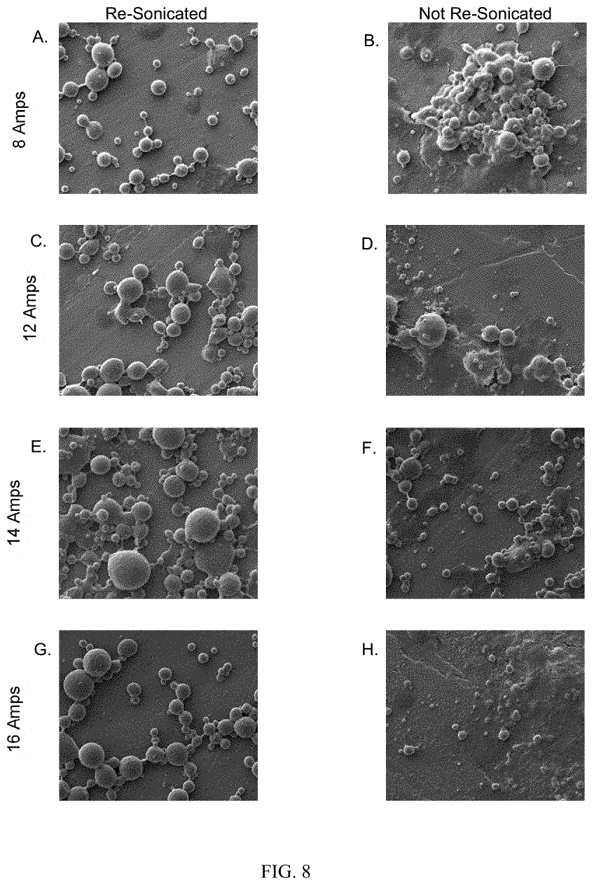

FIG. 8 shows silk particles resulting from sonication at different amplitudes and the impact of a re-sonication (15 amplitudes for 15 second) following PVA removal. Samples that were not re-sonicated yielded qualitatively larger aggregates. Re-sonication resulted in fewer aggregates. In some embodiments, re-sonication improves PVA removal and reduces particle aggregation.

TABLE 1
Average diameter and range of particle diameters for samples
sonicated in an ultrasonication bath, and with a ¼″
microtip at 4 amplitude, 8 amplitude, and 12 amplitude.

| | Average particle | Range |
|---|---|---|
| Sample | diameter (μm) | (μm) |
| Water bath | 8.61 | 1.0-33 |
| 4 Amps | 2.89 | 1.0-8.4 |
| 8 Amps | 2.52 | 1.0-7.5 |
| 12 Amps | 2.49 | 1.0-7.7 |

### Example 6 Encapsulation of Bovine Hemoglobin and PFOB in Silk Particles

To confirm PFOB and bovine Hb encapsulation in silk fibroin particles, silk fibroin particles were formed as described in Table 2 and observed for encapsulation efficiency.

TABLE 2
Gives a detailed description of the methods in formulating various samples of PFC and hemoglobin encapsulating particles.

| Control | Sample A | Sample B | Sample C | Sample D | Sample E + F | Sample F |
|---|---|---|---|---|---|---|
| PVA/Silk | 3% PFC in PVA | 5% PFC/EtOH + | PVA/Silk | PVA/Silk + PFC | PVA/Silk | PVA + Silk + |
| | | PVA + silk | | | | 400 mg hemoglobin/ |
| | | | | | | ml silk |
| waited two hours before sonication and stored the samples at 4° C. | | | | | | |
| in the interim | | | | | | |
| Sonicate at 12 | Sonicate at 12 | Sonicate at 12 amplitude 30 sec on ice | | | | |
| amplitude 30 sec | amplitude 1 sec | | | | | |
| on ice | on/1 sec 1 off | | | | | |
| | for 30 sec | | | | | |
| | Add silk, sonicate | | | | | |
| | for 30 sec | | | | | |

| Cast film in a Petri dish and keep under a Kimwipe partially uncovered under | Cast films in separate Petri |
|---|---|
| the fume hood for 48 hours | dishes and keep under a Kimwipe |
| | fully uncovered under the fume hood for 24 hours |
| | Peel off PVA/silk film and dissolve |
| | in 20 mL H₂O. |

Add Hemoglobin

| | |
|---|---|
| Dissolve film, sonicate at 16 Amp for 15 sec (optional), Centrifuged | Samples E and F combined and |
| at 4° C. for 20 min | centrifuged at 4° C. for 20 min |

| | | |
|---|---|---|
| resuspended the pellet in 5 mL H₂O | resuspended particles | resuspended the pellet in 5 mL H₂O |
| | in 5% PFC/EtOH | |
| | (Inverted on orbital | |
| | rocker for 30 minutes | |
| | to mix) | |

Sonicate at 16 Amp (or 15% power) for 15 sec to break apart particle aggregates
Store at 4° C. with a few drops of anti-anti (1x)
Optional re-sonication.

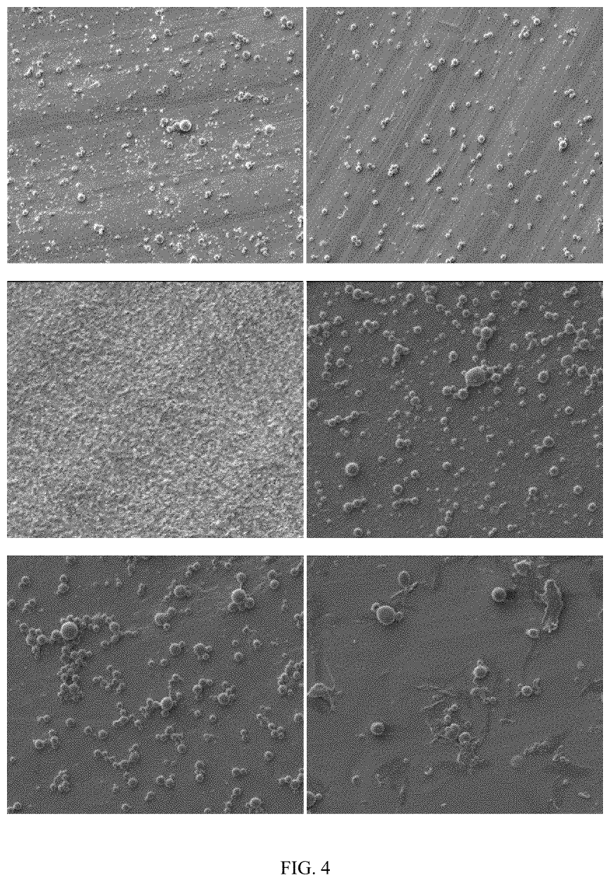

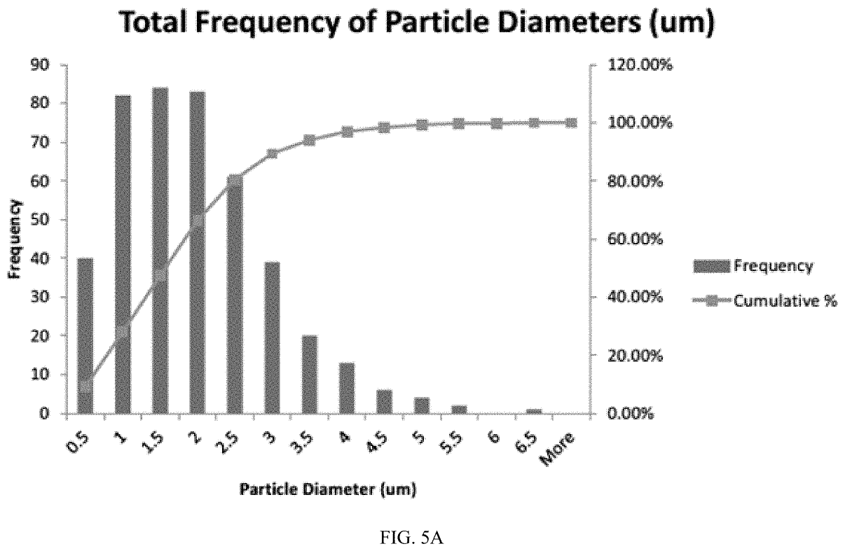

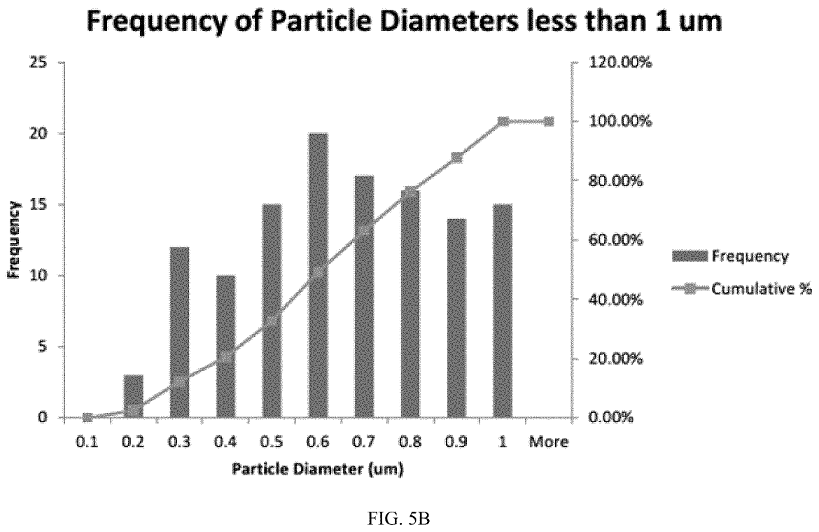

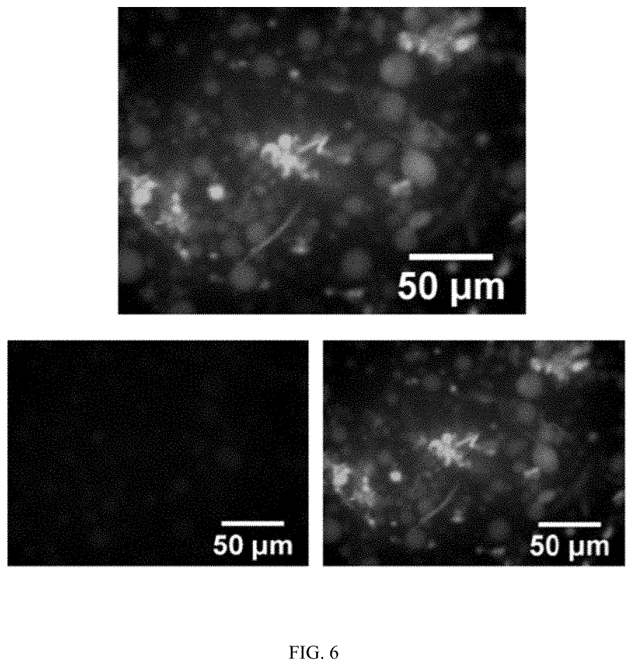

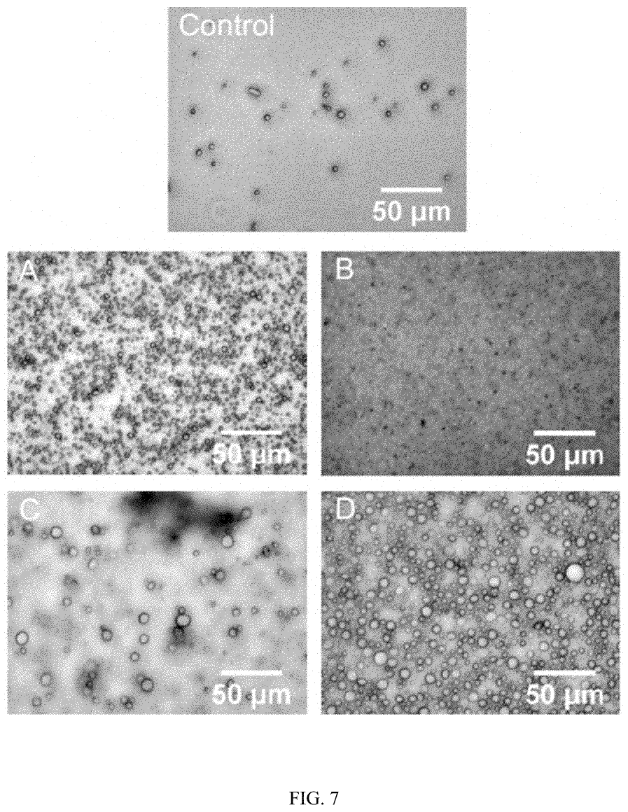

All samples were imaged under scanning electron microscopy (FIG. 4) and the size for the samples were analyzed. The polydispersity and confirmation of nanometer sized particles for Samples A, C, D, and E+F are graphically represented in FIG. 5. About 20% of particles formed were 1 micron or below in particle diameter. Particle diameters were not compared quantitively for the Ouzo method particles (Sample B) because no particles were formed in Sample B. Images were qualitatively analyzed under fluorescent microscopy to determine bovine hemoglobin encapsulation and under light microscopy to determine PFOB encapsulation (FIGS. 6 and 7). When comparing the size of the control sample to Sample C which had an addition of PFOB and ethanol at the end of the procedure, particle sizes were qualitatively smaller in sample C. To summarize, this experiment did not prove the aim that the Ouzo method would potentially form smaller silk particles that the control method.

A. Light and Fluorescent Microscopy Imaging. Microparticles were observed under 40× and 60× objective lenses on a Keyence (BZX Series). Silk-only particles and particles encapsulating PFOB were imaged using a phase contrast filter. Silk particles encapsulating bovine Hb were imaged under fluorescent microscopy (green). Due to the auto-fluorescence characteristic of silk, images were taken under blue light to differentiate particles from the encapsulated hemoglobin component. When imaged under 60×, settings were chosen as high sensitivity and the micron to pixel ratio for this, as well as the 40× objective, was 0.37744 μm/px

B. Staining of Hemoglobin Encapsulating Particles. A small volume (1 mL) of silk-bovine Hb particles was centrifuged in a 1.5 mL microcentrifuge tube until a pellet formed. Particles were rinsed in 1× PBS by slow resuspension and mixing using a P1000 micropipette before centrifuging again to obtain a pellet and decanting the supernatant. All rinsing between fixing, blocking, and staining with antibodies was performed as such for three rinses after each step. Fixing was performed using 10% Phosphate Buffered Formalin at room temperature for 20 minutes. Blocking was executed overnight at 4° C. using 1% bovine serum albumin (BSA) in 1× PBS with 5% Donkey Serum (Fisher Scientific, product no. 50-413-275). After blocking, primary unconjugated antibody, Anti-Hemoglobin Zeta Subunit (Sigma Aldrich AV-42231) diluted to 0.006 mg/mL using 0.2% BSA in 1× PBS, was used to resuspend the particles. This was inverted on a rocker for four hours. Secondary antibodies (Thermofisher, Alexa Fluor™ 488, product no. A21206) at 0.005 mg/mL in 0.2% BSA 1× PBS was used to resuspend the pellets and the particles were inverted in solution for two hours before imaging.

C. For scanning electron microscopy, samples were mounted and gold sputtered. Images of the particles were taken with an accelerating voltage of 5 kV and a working distance of 9.9-10.1 mm.

Bovine hemoglobin was successfully encapsulated in silk microparticles (FIG. 6) as indicated by fluorescence signal within silk fibroin particles. The presence of opaque particles in Sample A indicates encapsulation of PFOB (FIG. 7).

### Example 7. Measuring Oxygen Content in Silk Fibroin Particles

In addition to visual observation of particles, it is also possible to analyze oxygen content of the silk fibroin particles. Silk fibroin particles encapsulating an oxygen carrier are prepared as described. Either before or after encapsulation, the perfluorocarbon or hemoglobin is oxygenated. A solution of the silk fibroin particles is then analyzed by equipment capable of measuring oxygen content of the particles. Silk fibroin particles encapsulating oxygenated carrier are then compared with empty silk fibroin particles or silk fibroin particles encapsulating oxygen carrier which is not oxygenated. An increase in oxygen detected in silk fibroin particles encapsulating oxygenated carrier relative to control samples indicates the presence of the oxygen carrier in the silk fibroin particles and the ability of the oxygen carrier in the particles to carry oxygen.

### Example 8. Formation of Silk Fibroin Particles Encapsulating Hemoglobin

Silk particles were formed via phase separation with polyvinyl alcohol (PVA) as described in example 4 and Rockwood et al. (Rockwood et al. “Materials fabrication from Bombyx mori silk fibroin.” Nat Protoc. 2011; 6(10):1612-31).

Preparation of silk fibers: Empty cocoons were boiled for 30 minutes, 60 minutes, or 90 minutes in 0.02 M Na₂CO₃. Boiled fibers were then rinsed 3 times in water for 20 minutes each. Excess water was removed and the washed fibers were dried. 9.3M LiBr was added to the dried silk fibers and incubated at 60° C. for 4 minutes. The treated fibers were then dialyzed against water for 48 hours. Silk fibers were then collected by centrifugation and stored at 4° C.

Phase Separation: For phase separations, samples were prepared as described above at silk fibroin concentrations of 20 mg/mL, 50 mg/mL, and 70 mg/mL. At each silk concentration, silk fibroin to PVA ratios of 1:1, 1:3, 1:4 and 1:5 were used. Phase separation was induced via probe sonication, using 0%, 8%, 12%, 16%, 25%, or 40% power. In some samples, bovine hemoglobin was incorporated into the particles at concentrations of 0.25, 0.5, 1.0. and 1.5 mg/mL by reconstituting substrate powder hemoglobin in ultrapure water and combining with the silk solution prior to sonication. For some samples, the silk particles were sonicated a second time at 15% power for 15 seconds.

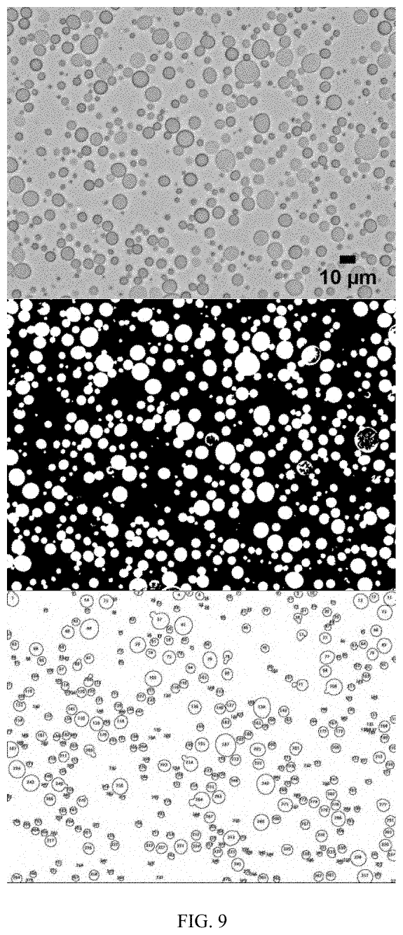

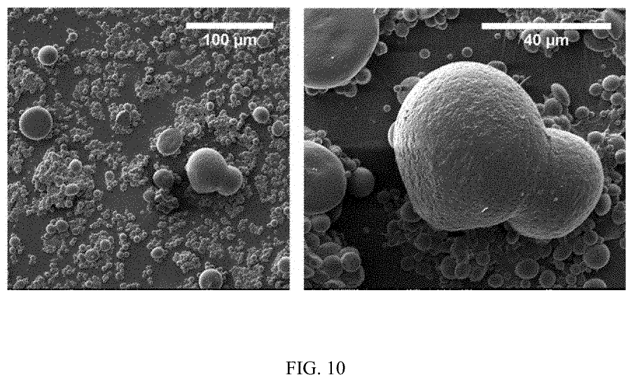

Particle characterization: Samples were assessed for particle size, stability, and morphology using light microscopy (FIG. 9), dynamic light scattering (DLS), and scanning electron microscopy (SEM; FIG. 10). Particles were analyzed by light microscopy (60×) using the ImageJ “Analyze Particles” tool (NIH). The “Analyze Particles” tool allows for the removal of touching and out of focus particles from analysis (slide 1). Dynamic light scattering was used to estimate particle size in the nanometer range and to provide a measure of particle stability. Scanning electron microscopy was used to view particle morphology and size for particles smaller than could be observed using light microscopy. Immunofluorescent imaging was performed to confirm colocalization of the hemoglobin in the silk particles.

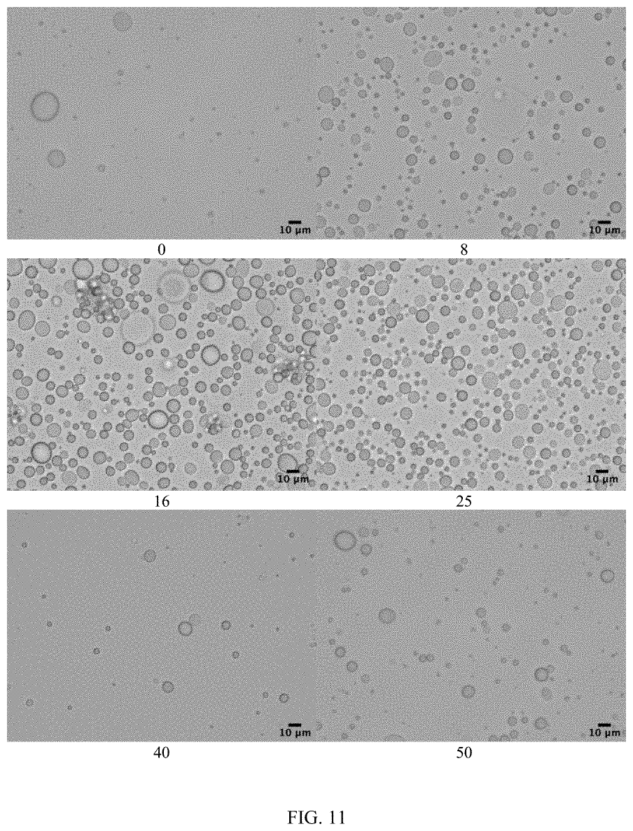

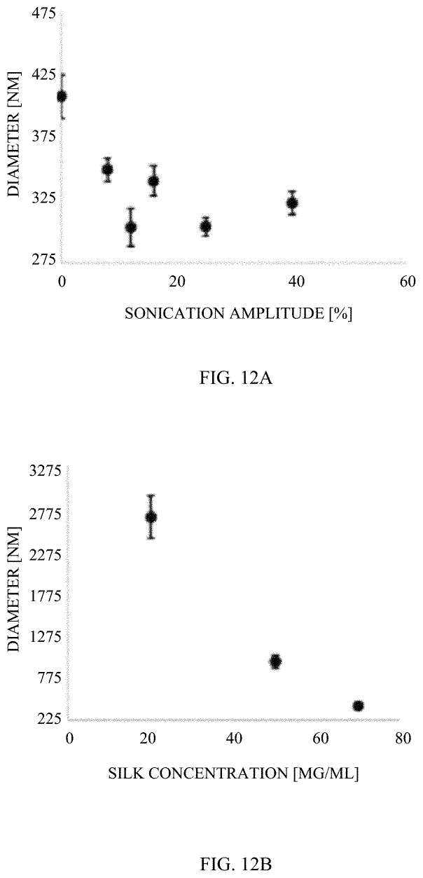

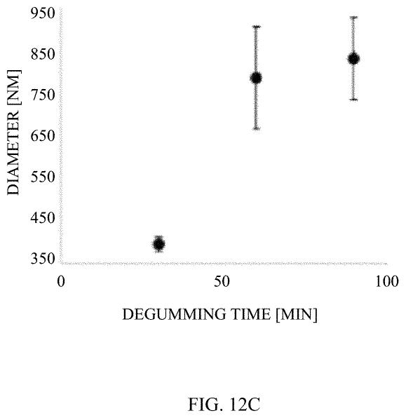

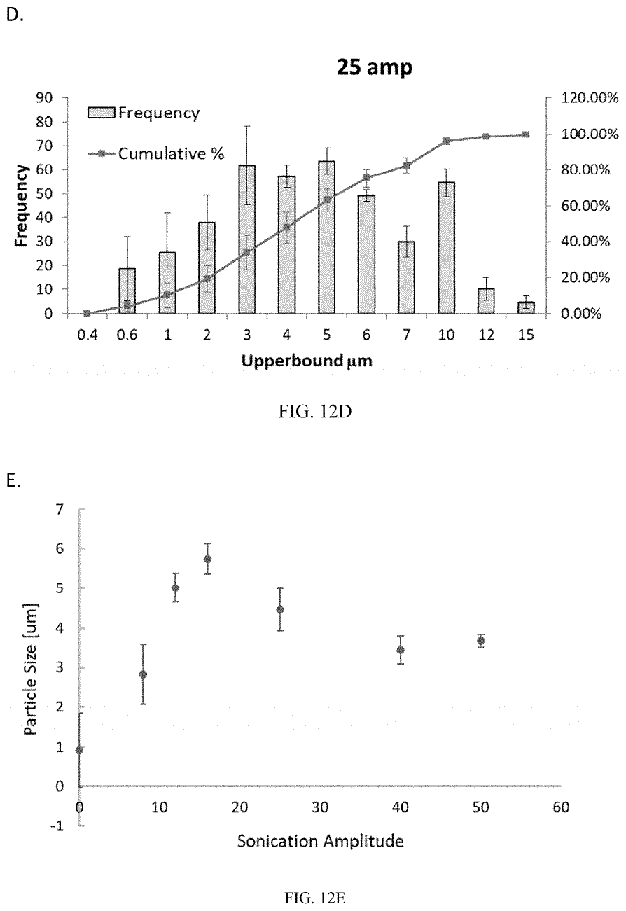

Silk fibroin particles were present for all sonication amplitudes tested (FIG. 11). Increased sonication amplitude and increasing silk concentration led to smaller particle size (FIG. 12A, B). Increasing silk degumming time led to increased particle size (FIG. 12C). Silk to PVA ratio was not observed to have a substantial effect on particle size at the ratios tested. Moderate to high levels of polydispersity were observed across all samples. Samples were then filtered to narrow the polydispersity of the particles. Bovine hemoglobin was observed to be incorporated in particles (FIG. 1D). No significant changes in particle size were observed as a function of the hemoglobin inclusion.

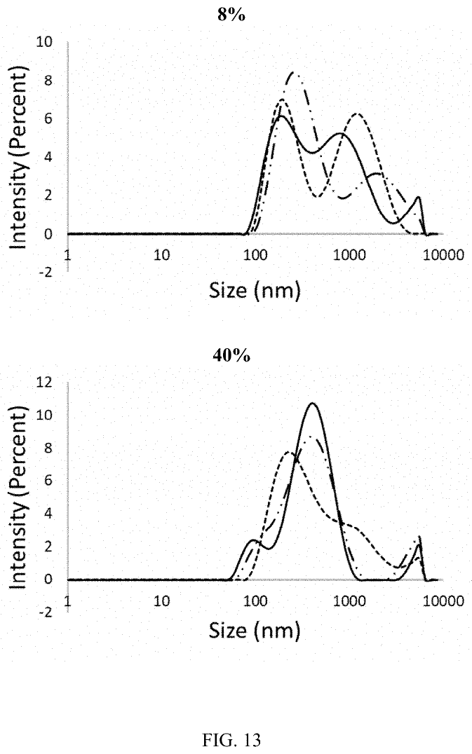

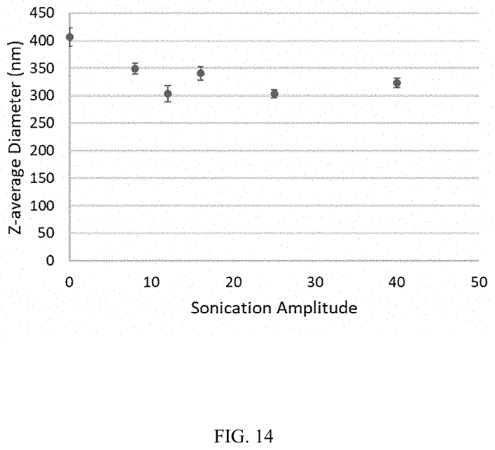

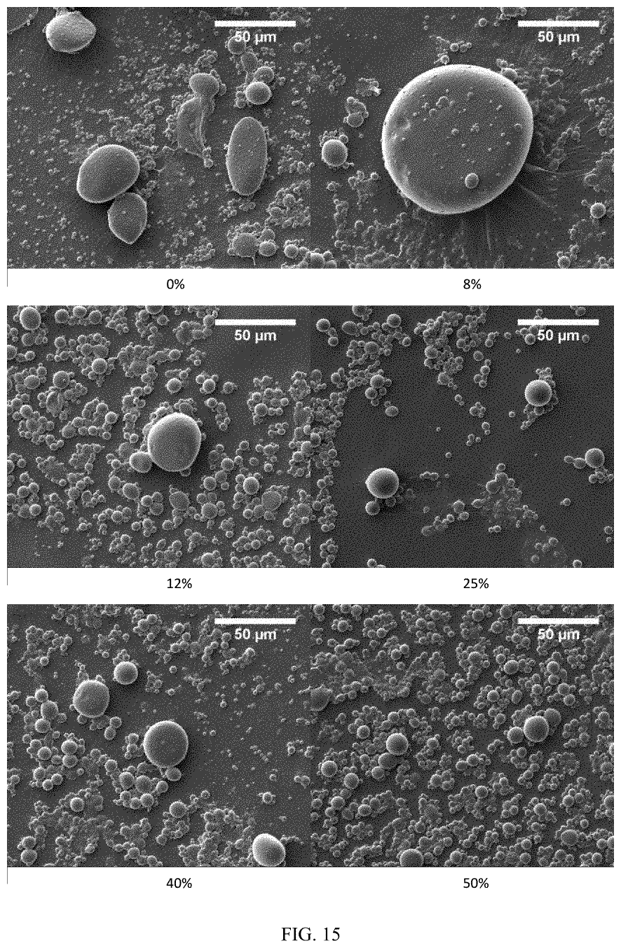

Particle size distribution (PSD) for various sonication amplitudes is shown in FIG. 12. Frequency of various particle size following sonication of 25% is shown in FIG. 12D. Average particle size at various sonication amplitudes is shown in FIG. 12E. Surprisingly, the peak particle size was maximal at about 16%, with sonication and higher and lower amplitudes resulting in lower average particle sizes. Across the sonication range DLS further showed that the signal for large particles (>1 um) decreased with increasing sonication amplitude. Further, average particle size decreased with increasing sonication amplitude (FIGS. 13-14). Scanning electron microscopy showed that sonication at low amplitude resulted in the presence of large, non-spherical particles (FIG. 15). The presence of large particles decreased with increasing sonication amplitude (FIG. 15).

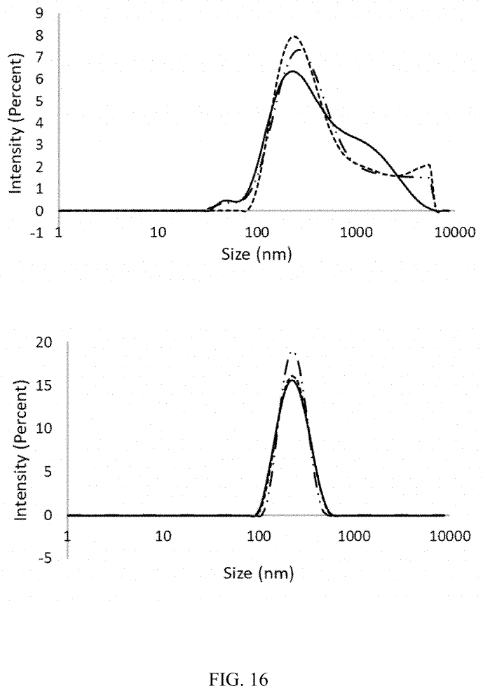

To narrow the range of particle sizes, the silk particles with sonicated a second time (15% for 15 seconds) filtered through a 650 nm cutoff filter. Filtration significantly reduced particle size dispersity and yielded a sample with an average size of about 200 nm and low polydispersity (FIG. 16). Scanning electron microscopy showed the sample contained debris from the larger particles.

Immunofluorescent imaging confirmed colocalization of the hemoglobin in the silk particles.

A COMSOL model (e.g., 2D Reaction-Diffusion COMSOL model) can be used to analyze/predict the oxygen dynamics of the silk particles. Oxygen delivery profiles can be measured using an in vitro perfusion flow system equipped with PreSens® oxygen flow sensors.

1. A blood substitute comprising an oxygen carrier encapsulated in silk fibroin particles.

2. The blood substitute of claim 1, wherein the silk fibroin particles are less than 1000 nm in diameter.

3. The blood substitute of claim 2, wherein the silk fibroin particles are 200-500 nm in diameter.

4. The blood substitute of any one of claims 1-3, wherein the oxygen carrier is a hemoglobin.

5. The blood substitute of claim 4, wherein the hemoglobin is a Root effect hemoglobin.

6. The blood substitute of claim 5, wherein the hemoglobin comprises a teleost hemoglobin.

7. The blood substitute of claim 6, wherein the teleost hemoglobin comprises a salmon hemoglobin or a trout hemoglobin.

8. The method of claim 4, wherein the hemoglobin comprises a mammalian hemoglobin.

9. The method of claim 8, wherein the mammalian hemoglobin comprises human hemoglobin, bovine hemoglobin, horse hemoglobin, pig hemoglobin, sheep hemoglobin, or goat hemoglobin.

10. The blood substitute of any one of claims 4-9, wherein the hemoglobin is a recombinant hemoglobin or modified hemoglobin.

11. The blood substitute of any one of claims 1-3, wherein the oxygen carrier is a perfluorocarbon.

12. The blood substitute of claim 11, wherein the perfluorocarbon is perfluorooctyl bromide.

13. The blood substitute of any one of claims 1-12, wherein the silk fibroin is obtained from raw silk by degumming the raw silk for 15 minutes to 2 hours.

14. The blood substitute of claim 13, wherein the silk fibroin is obtained from raw silk by degumming the raw silk for about 60 minutes.

15. The blood substitute of any one of claims 1-14, wherein the silk fibroin particles are formed by a lipid micelle encapsulation method or a phase separation method.

16. A method for forming silk fibroin particles encapsulating an oxygen carrier comprising:
(a) adding silk fibroin and the oxygen carrier to a polyvinyl alcohol (PVA) and water solution to form a mixture;
(b) sonicating the mixture;
(c) casting the solution in a container and drying the solution to form a film; and
(d) dissolving the film in an aqueous solution to form silk fibroin particles and sonicating silk fibroin particles, thereby forming the silk fibroin particles encapsulating the oxygen carrier.

17. The method of claim 16, further comprising obtaining the silk fibroin from raw silk by degumming the raw silk for 15 minutes to 2 hours prior to step (a).

18. The method of claim 16, further comprising obtaining the silk fibroin from raw silk by degumming the raw silk for about 60 minutes prior to step (a).

19. The method of any one of claims 16-18, wherein the oxygen carrier is a hemoglobin.

20. The method of claim 19, wherein the hemoglobin is a Root effect hemoglobin.

21. The method of claim 20, wherein the hemoglobin comprises a teleost hemoglobin.

22. The method of claim 21, wherein the teleost hemoglobin comprises a salmon hemoglobin or a trout hemoglobin.

23. The method of claim 19, wherein the hemoglobin comprises a mammalian hemoglobin.

24. The method of claim 23, wherein the mammalian hemoglobin comprises human hemoglobin, bovine hemoglobin, horse hemoglobin, pig hemoglobin, sheep hemoglobin, or goat hemoglobin.

25. The method of any one of claims 19-24, wherein the hemoglobin is a recombinant hemoglobin.

26. The method of any one of claims 16-18, wherein the oxygen carrier is a perfluorocarbon.

27. The method of claim 26, wherein the perfluorocarbon is perfluorooctyl bromide.

28. The method of any one of claims 16-27, wherein the PVA and water solution contains 1-10% PVA.

29. The method of claim 28, wherein the PVA and water solution contains 4-5% PVA.

30. The method of any one of claims 16-29, wherein the ratio of silk fibroin to the PVA and water solution is 0.1-10:100 weight to volume.

31. The method of claim 30, wherein the ratio of silk fibroin to the PVA and water solution is 1:100 weight to volume.

32. The method of any one of claims 16-29, wherein the ratio of silk fibroin to PVA is 1:1-25 weight to volume.

33. The method of claim 32, wherein the ratio of silk fibroin to the PVA 1:4 to 1:5 weight to volume.

34. The method of any one of claims 16-33, wherein sonicating the mixture at (b) comprises (i) sonicating for 5-60 seconds at 4-16 amplitude using a probe sonicator or (ii) sonicating for 5-60 seconds at 8-50% power using a probe sonicator.

35. The method of claim 34, wherein sonicating the mixture at (b) comprises (i) sonicating for 30 seconds at 12 amplitude using a probe sonicator or (ii) sonicating for 30 seconds at 25% power using a probe sonicator.

36. The method of any one of claims 16-35, wherein sonicating the mixture at (d) comprises (i) sonicating for 5-60 seconds at 4-16 amplitude using a probe sonicator or (ii) sonicating for 5-60 seconds at 16-40% power using a probe sonicator.

37. The method of claim 36, wherein sonicating the mixture at (d) comprises (i) sonicating for 30 seconds at 12 amplitude using a probe sonicator or (ii) sonicating for 15 seconds at 15% power using a probe sonicator.

38. The method of any one of claims 13-37, wherein the silk fibroin particles are further re-sonicated after step (d).

39. The method of any one of claims 16-38, wherein the silk fibroin particles are less than 1000 nm in diameter.

40. The method of any one of claims 16-38, wherein the silk fibroin particles are 200-500 nm in diameter.

41. A method for forming silk fibroin particles encapsulating an oxygen carrier comprising:
(a) forming a film of phospholipid on a surface;
(b) adding silk fibroin and the oxygen carrier in an aqueous solution to the film thereby forming lipid micelles;
(c) subjecting the sonicated lipid micelles to at least one freeze-thaw cycle;
(d) lyophilizing the lipid micelles;
(e) washing the lyophilized lipid micelles in methanol to yield the silk fibroin particles encapsulating the oxygen carrier.

42. The method of claim 41, wherein the lipid micelles are sonicated prior to the freeze thaw cycle at (c) and/or prior to lyophilizing at step (d).

43. The method of claim 42, wherein the lipid micelles are (i) sonicated for 5-60 seconds at 4-16 amplitude using a probe sonicator or (ii) sonicated for 5-60 seconds at 8% to 50% power using a probe sonicator.

44. The method of claim 43, wherein the lipid micelles are (i) sonicated for 30 seconds at 12 amplitude using a probe sonicator or (ii) sonicated for 30 seconds at 25% power using a probe sonicator.

45. The method of any one of claims 41-44, wherein the silk fibroin particles encapsulating the oxygen carrier are dissolved in aqueous solution after step (e).

46. The method of claim 45, wherein the silk fibroin particles encapsulating the oxygen carrier dissolved in aqueous solution are sonicated.

47. The method of claim 46, wherein the silk fibroin particles are (i) sonicated for 5-60 seconds at 4-16 amplitude using a probe sonicator or (i) sonicated for 5-60 seconds at 15% power using a probe sonicator.

48. The method of claim 47, wherein the silk fibroin particles are (i) sonicated for 30 seconds at 12 amplitude using a probe sonicator or (i) sonicated for 15 seconds at 15% power using a probe sonicator.

49. The method of any one of claims 41-48, further comprising obtaining the silk fibroin from raw silk by degumming the raw silk for 15 minutes to 2 hours prior to step (a).

50. The method of any one of claims 41-48, further comprising obtaining the silk fibroin from raw silk by degumming the raw silk for about 60 minutes prior to step (a).

51. The method of any one of claims 41-50, wherein the oxygen carrier is a hemoglobin.

52. The method of claim 51, wherein the hemoglobin is a Root effect hemoglobin.

53. The method of claim 52, wherein the hemoglobin comprises a teleost hemoglobin.

54. The method of claim 53, wherein the teleost hemoglobin comprises a salmon hemoglobin or a trout hemoglobin.

55. The method of claim 51, wherein the hemoglobin comprises a mammalian hemoglobin.

56. The method of claim 55, wherein the mammalian hemoglobin comprises human hemoglobin, bovine hemoglobin, horse hemoglobin, pig hemoglobin, sheep hemoglobin, or goat hemoglobin.

57. The method of any one of claims 51-56, wherein the hemoglobin is a recombinant hemoglobin.

58. The method of any one of claims 41-50, wherein the oxygen carrier is a perfluorocarbon.

59. The method of claim 58, wherein the perfluorocarbon is perfluorooctyl bromide.

60. The method of any one of claims 41-59, wherein the phospholipid comprises 1,2-dioleoyl-sn-glycero-3-phosphocholine or dipalmitoylphosphatidylcholine.

61. The method of any one of claims 41-60, wherein the silk fibroin particles are less than 1000 nm in diameter.

62. The method of any one of claims 41-60, wherein the silk fibroin particles are 200-500 nm in diameter.

63. A method of treating a subject in need of increased oxygen supply to a tissue or organ comprising administering to the subject a composition containing the blood substitute of any one of claims 1-15.

64. The method of claim 63, wherein the subject is suffering from anemia, hemorrhage, acute loss of blood due to trauma or surgery, ischemia, hypoxia, or septic shock.

65. A method of supplying an ex vivo tissue or organ with oxygen comprising perfusing the tissue or organ with a composition containing the blood substitute of any one of claims 1-15.

66. A silk fibroin nanoparticle encapsulating an oxygen carrier made by the method of any one of claims 16-60.
